(19) United States
(12) Patent Application Publication
(10) Pub. No.: US 2003/0208135 A1
(43) Pub. Date: Nov. 6, 2003

(54) Method and system for biopsy and analysis of body tissue

(76) Inventors: Kenneth Bloom, Irvine, CA (US); Seth A. Stabinsky, Pleasanton, CA (US)

Correspondence Address: Crockett & Crockett, Suite 400, 24012 Calle De La Plata, Laguna Hills, CA, 92653, US

(21) Appl. No.: 10/137,910

(22) Filed: May 3, 2002

(51) Int. Cl. A61B 10/00

(52) U.S. Cl. 600/564

(57) ABSTRACT

Methods and systems for performing biopsies and diagnosing tumors and suspect masses within the body. A biopsy sample is frozen while still in the body of a patient, then removed from the patient, stored frozen, and subject to IHC, microarray or other analysis to determine the amount, type or presence of signaling substances within the tumor or suspect mass.

### FIELD OF THE INVENTIONS

The methods and systems described below relate to the fields of biopsy and pathology.

### BACKGROUND OF THE INVENTIONS

Biopsy is a common procedure for obtaining a sample of tissue from a patient for analysis. When a patient and doctor suspect that a portion of the patient's body may be diseased, this suspicion is confirmed or dissipated by taking a small sample, through one of many biopsy procedures, and analyzing the sample through one of many pathology tests. A typical example of biopsies and their associated tests is breast biopsies and staining techniques such as Hematoxylin and Eosin or immunohistochemistry techniques such as with HER-2/Neu detection. These tests are intended to determine if a suspect tumor in a woman's breast is cancerous or benign and to predict its aggressiveness. Tumors and masses elsewhere in the body, such as the brain, liver, lung, colon, or head and neck, may be sampled and analyzed with similar procedures.

When doctors are trying to diagnose breast cancer in a patient, samples taken from the patient's breast are delivered to a pathologist for analysis. The sample is obtained with a biopsy procedure, which may be performed in many ways, but is typically done with a biopsy gun which is inserted into the breast through a small puncture. After the sample is harvested, it is typically placed in 10% buffered formalin or alcohol and transmitted to the pathologist. Subsequent analysis by the pathologist may take place hours or days after the biopsy. To determine whether the sample is cancerous or benign, the pathologist merely compares the physical appearance of the sample (under a microscope) to the known physical appearance of known cancerous samples. The pathologist evaluates the presence of certain cells, the distribution and density of cells, and the arrangement of the cells in the sample (what we call the cellular architecture), and based on this visual inspection, the pathologist decides whether the sample tissue is cancerous or not. Thus, the parameters that pathologists have traditionally used to characterize cancers stem from their static appearance under the microscope. Additionally, descriptions of various parameters such as tumor type, size, grade, nuclear grade, mitotic count, lymphovascular invasion, necrosis and the presence of loco-regional or distant metastases are used by oncologists to decide what kind of cancer is in the sample and which of various treatments would be most likely to cure the patient. Though it is the current standard for diagnosis, observation of the architectural patterns observed in preserved tissue under the microscope give little insight into the metabolic function that was occurring within that tissue prior to its excision, preparation, and staining, and this information may be quite valuable for diagnosis and selection of treatment.

Over the last several years, pathologists have developed immunohistochemistry (IHC) techniques to help analyze potentially cancerous tissue. IHC is used to detect the presence of specific membrane, cytoplasmic, and/or nuclear receptors (proteins) that may be predictive of both the aggressiveness of a given tumor and of specific therapies that might be effective against the cancer. The presence of Estrogen and Progesterone receptors in breast cancer, for example, generally implies a better prognosis and suggests that hormonal therapy may be helpful. On the other hand, the over-expression of HER-2/Neu is considered a sign of a more aggressive tumor that may not be susceptible to hormonal therapy. HER-2/Neu is an epidermal growth factor receptor that is found on all cell membranes, but in some breast cancer tissue it is over-expressed or amplified, as scientists refer to abnormally high levels of production substances by body tissue. Thus, HER-2/Neu will be present in all biopsied tissue, but will be present in unusual quantities in cancerous tissues if the cancer is of a specific type. If it is known that HER2/Neu is over-expressed, doctors will know first that the tumor is cancerous and aggressive, and second that the cancer is likely to be highly susceptible to treatment with chemotherapy augmented with trastuzumab (also known as Herceptin). Herceptin is an antibody-based drug that is targeted to attack the cell-surface receptor that is overproduced by these so called “HER-2/Neu positive” patients.

Unfortunately, about 30% of the time, when conventional IHC laboratory methods would lead one to believe that a breast cancer is a low risk, non-aggressive cancer (that is, no over-expression of HER2 is detected), it turns out to be aggressive and it kills the patient, and about 30% of the time that a tumor is predicted to be aggressive (over-expression of HER2 is detected) it turns out to be non-aggressive and the patient exhibits long-term survival. This means that some patients get very expensive treatment that is useless, and some patients who might survive with the treatment do not get it and die as a result.

Despite its promise, IHC for HER-2/Neu testing is not reliable. One reason for the unreliability in IHC testing is that formalin fixation causes cross-linking of proteins in the region of the cell surface receptors (antigens) that bind the HER-2/Neu antibody used in the IHC test. Thus, the HER-2/Neu levels are altered by the fixation technique. Cavalier tissue handling, particularly prolonged exposure to room temperature prior to placement in formalin, can also exacerbate this reaction. Because of such cross-linking, an antigen retrieval process is a necessary part of IHC, but retrieval is not complete, and variation in retrieval is not predictable. Excessive or insufficient fixation or antigen retrieval can cause false positive or negative results, as can variations in tissue handling.

As stated, there can be overlap in the levels of HER-2/Neu expression seen in normal breast tissue (or less aggressive cancers) and those seen in aggressive cancers. These confusing results are the direct result of a lack of sensitivity in testing available today. But because HER-2/Neu over expression is actually due to amplification of a gene at the DNA level (i.e. There are many copies of a gene that should only have two copies in each cell), another test, called fluorescent in situ hybridization (FISH), which identifies the presence of the abnormal DNA can be used for the interpretation of indeterminate IHC results. Unfortunately, errors at the DNA level such as gene amplification are a very rare cause of overproduction of cellular proteins that cause normal cells to become cancerous ones. It is an infinitely more common phenomenon to have abnormal protein production caused by the presence of abnormal or “mutant” messenger ribonucleic acid (mRNA). Many different mutant forms of mRNA can exist in a cell that is either destined to become or already is malignant. These mRNA lead to the production of proteins that drive abnormal cell growth. The mRNA need not be expressed in high concentrations at all. Such mutant mRNA in low concentration is said to have “low copy numbers.” Laboratory techniques such as polymerase chain reaction (PCR) are required to amplify (make many copies) of the mRNA so that it can be detected in such cases.

Some breast cancers exhibit the over-expression of an epidermal growth factor receptor (EGFR) called HER-1. Phosphorylation of this receptor is known to be a mechanism by which cancer cells become more aggressive, grow, and spread. This phosphorylation is detected by the over-expression of mutant mRNA. The drug ZD1839 (also known as Iressa) has been demonstrated to be effective against such cancers. It is another antibody-based drug that targets the phosphorylation site of an epidermal growth factor receptor (EGFR) and is only effective in those tumors where phosphorylation is taking place. It is anticipated that Iressa will be extremely expensive (approximately $2,500 per week for many weeks) when it is eventually approved in the United States. Therefore, knowing which patients are most likely to benefit from treatment with ZD1839 and which patients could benefit is important from not only a clinical standpoint, but also from an economic one.

Unfortunately, no test is currently available to predict the activity and signaling substances characteristic of Iressa-susceptible cancers. The main reason why no test is available to determine whether a given tumor is phosphorylating the HER-1 receptor site is that the signaling mRNA degrades nearly instantaneously after excision of a biopsy sample. Cellular mRNA levels are exquisitely sensitive to their environment, and mRNA may be present within a cell for only a few seconds after excision. Thus, so little of the signaling mRNA is available in the sample when it finally arrives at a lab for testing that tests cannot determine whether there is or was an unusually high level of the signaling mRNA in the biopsied tissue or, for that matter, which mutant mRNA was present.

Iressa is not the only drug being developed for therapies targeted against a specific signal that causes a cell to become cancerous. There are dozens of such drugs undergoing evaluation. One other example of such a drug class is “anti-angiogenesis” compounds. Cancer cells over-produce angiogenesis factors that stimulate local growth of blood vessels. Such angiogenesis is thought to be a major mechanism that allows metastases to occur. It is also a normal, physiologic response for benign cells that are hypoxic (low on oxygen) to release mRNA. The mRNA, in turn, signals the cells to produce proteins that are part of long pathway that leads to eventual angiogenesis. During traditional tumor biopsy or excision, the tissue becomes hypoxic due to excision, and may produce angiogenic factors in quantities that mask or mimic the cancer-induced over-production. Thus, finding an accurate way of assessing types and levels of mRNA that were present within normal and cancerous cells prior to their detachment from their native oxygen supply will be critical for the development of anti-angiogenesis drugs.

Thus, different types of breast cancer (and other cancers as well) respond differently to different treatments. Some breast cancers which express Estrogen receptors (ER) or progesterone receptors (PR) or exhibit an over expression of GATA3, are best treated with hormonal therapy, while other types, such as those that over-express HER-2/Neu or mutant HER-1 mRNA, are best treated with Herceptin or Iressa, respectively. As increasing numbers of pharmacologic interventions are developed that target specific cellular pathways, optimization of sample collection will be required in order to accurately quantify differences between cancer cells and benign cells. In some cases, such as where mutant mRNA is being sought, progress will be severely hampered until such optimal sample preservation is accomplished easily. Today, IHC works well when high levels of a receptor are present, but in the case of “subtle over-expression”, there is room for improvement in the biopsy process. Thus, some types cancers may be differentiated by abnormal amounts, types or characteristics in signaling substances, such as DNA, mRNA, or proteins, but current biopsy techniques permit degradation of some signaling substances to the point where little or no information remains at the time the samples are analyzed.

### SUMMARY

The methods and systems described below permit biopsy samples to be taken, and subsequent testing to be accomplished, in such a manner that signaling substances are preserved in the tissue. Thus, rapidly degrading signaling substances, such as mRNA and growth factors such as HER-2/Neu can be detected with accuracy, and the appropriate therapy may be selected with knowledge as to the susceptibility of the diseased tissue to the various available cancer treatments. Also, rapidly degrading downstream regulators, such as AKt and PI3K can be detected with accuracy never before possible. Appropriate therapy may then be selected to target cancer cell behavior much more specifically.

In situ cryopreservation biopsy will eliminate any issues with cross-linking, because fresh frozen samples are used instead of formalin-preserved tissue. This will permit standardization of harvesting techniques allowing much better comparisons across laboratories. Standardization will make multi-center clinical trial results more reliable and allow more laboratories to perform complicated analyses with more confidence. Local hospital laboratories will be able to carry out complex tissue analyses with results that are as accurate and precise as much larger commercial laboratories.

In situ cryopreservation biopsy will also eliminate much of the mRNA degradation inherent in tissue sampling. Because the sample is frozen very rapidly, before its blood supply is cut off, and because it will be kept stable in the frozen state until the receiving laboratory is ready to perform an enzyme extraction on it, the mRNA content of the tissue will more accurately reflect the mRNA content of the tissue as it was in vivo.

### DETAILED DESCRIPTION OF THE INVENTIONS

The first step in the biopsy and testing method is a biopsy of a suspect mass under conditions that preserve quickly degrading components of the tissue in the suspect mass. In particular the biopsy will take place under conditions that will preserve the cells in a state as close to their in vivo environment as possible. When used to test a breast tumor or other suspect mass in the breast, the tissue components of concern include many different receptors, including but not limited to HER-2/Neu (also referred to as c-erbB-2 or ERBB2) and HER-1, as well as both mutant and normal mRNA. Using devices such as the Sanarus™ cryobiopsy device, or any other means of freezing body tissue in situ, a breast tumor is frozen before it excised from the surrounding tissue. After the cryobiopsy device is inserted into the breast, and the freezing segment of the cryobiopsy probe is inserted into the tumor, the tissue is frozen and cooled to cryoablative temperatures, in the range of +15 to −75 F. (−10 to −60° C.), thereby assuring cryopreservation. The biopsy specimen is thus frozen while it is still inside the body, prior to interrupting its blood supply during excision. Standard surgical techniques may be used to excise the tissue, or the cryobiopsy probes of our co-pending application Ser. No. 09/690,321 may be used. A tumor may also be excised in its entirety, using a cryoprobe, which is frozen within the tumor, to manipulate the tumor and facilitate its surgical removal. In such a case, the iceball, which is created by the cryoprobe, provides a template for excision and defines the boundaries of the tissue to be excised.

When the frozen tissue is removed from the body, it is placed in liquid nitrogen (−70° C.) or some other cooling system, so that it may be maintained in its frozen state until it can be analyzed. (The cryobiopsy device, cryoprobe, or a portion thereof may be deposited, with the sample still affixed, into the cooling system. The sample may, instead, be separated from the cryoprobe device in the operating room before it is deposited into the cooling system.) The biopsy sample is thus maintained without degradation of signaling substances such as mRNA and HER-2/Neu and others.

To detach the specimen from the cryoprobe or cryobiopsy needle, the laboratory technician will dip the frozen specimen into the enzymatic solution used to break down the cells and expose the mRNA or receptors. The tissue will thaw within the liquid and slip off of the probe or needle. The frozen tissue sample may also be removed from the cryoprobe in the manner specified in our co-pending application Ser. No. 09/690,321. If an entire tumor is excised, it may be stored in liquid nitrogen in whole or it may be sliced and stored in slices.

Whether the samples are analyzed locally, in the same facility where the biopsy samples are achieved, or remotely in laboratories specially dedicated to analysis, they are thawed and processed, and subjected to testing such as PCR and IHC, which will detect signaling substances such as mRNA and HER-2/Neu expression. Testing may be accomplished using microarray kits, also referred to as gene chips, which quickly identify not only the presence and quantity of numerous genes and mRNA in the tissue, but also whether they are up-regulated or down-regulated (“turned on or turned off). Many targeted cancer therapies in the future will be based on altering the activity of cancers at this level. Available kits include ones manufactured by companies like Affymetrix, but they can also be custom made by laboratories carrying out the testing. Automated devices such as the “Light Cycler” from Roche are available to optimize the retrieval of mRNA, but they cannot retrieve what has already degraded between tissue harvesting and arrival in the laboratory. Therefore, the accuracy of instruments like this one can only be enhanced by better preserving the mRNA with cryopreservation biopsy.

Typically, after biopsy tissue arrives in the laboratory, it is placed into an enzyme solution where it is cut up and undergoes enzymatic extraction of the mRNA. Currently, the ideal specimen for such analyses is one that was freshly frozen in liquid nitrogen soon after removal in the operating room, but as discussed above, even in these samples much of the mRNA may be different than that which was present when the tissue was in the patient with an intact blood supply. In situ cryopreservation biopsy will ensure that the mRNA levels will remain stable in the tissue after its excision from the patient. Based on the measured amount of the signaling substance, the appropriate treatment (the treatment most likely to successfully treat the tumor) for the tumor may be selected. Selection is based on empirical studies which currently show that expression of substances over a certain threshold are indicative of the susceptibility of the tumor to certain treatments, and perhaps indicative of resistance to certain other treatments. For some combinations of substance expression and treatment, the thresholds are known, as described above. (Other combinations will be discovered in the practice of this biopsy method because it preserves information that has previously been obliterated by the routine sampling technique.) Inter-laboratory variation is very common when performing IHC. Microarray analysis would be even more variable across sites, due to unpredictable effects of the local environment and tissue handling. With the standardization possible using our new procedure, site-specific variations in the measured signaling substances should be eliminated so that borderline measurements which would indicate susceptibility to a treatment are not masked by site-specific variations in sampling and handling techniques.

Some of the treatments described above are described in terms of the well-known brand names of the drugs used, and these are the most concise identifications of the compounds suitable for use in the diagnosis and treatment method. However, corresponding chemical compounds and generic versions of these drugs may be used. The diagnosis and treatment method has been described and illustrated in relation to breast cancer, but can be applied to any cancer or disease throughout the body in which quickly degrading signaling substances exist and are of diagnostic value. While the cryobiopsy device is well suited to excising the biopsy sample from the body, other means for excising the biopsy sample may be used. Also, while microarray kits and IHC have been discussed as tests which benefit from the method, other means for testing the biopsy samples may be used, and it is expected that additional means will be devised in the future which will benefit from the biopsy method. While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A method for diagnosing breast tumors comprising:
freezing a biopsy sample within a tumor;
excising the frozen biopsy sample from the tumor and from the body, after freezing;
testing the biopsy sample for the presence of a signaling substance which indicates the susceptibility of the tumor to a course of treatment.

2. A method for diagnosing breast tumors comprising:
freezing a biopsy sample within a tumor;
excising the frozen biopsy sample from the tumor and from the body, after freezing;
testing the biopsy sample for the presence of a signaling substance which indicates the prognosis of the tumor.

3. The method of claim 1 or 2, wherein the testing comprises:
testing the biopsy sample with a microarray to determine the amount of mRNA in the biopsy sample.

4. The method of claim 1 or 2, wherein the testing comprises:
testing the biopsy sample with a microarray to determine the type of mRNA in the biopsy sample.

5. The method of claim 1 or 2, wherein the testing comprises:
testing the biopsy sample with a microarray to determine the presence or amount of mutant mRNA in the biopsy sample.

6. The method of claim 1 or 2, wherein the testing comprises:
testing the biopsy sample with a microarray to determine the amount of HER-2/Neu in the biopsy sample.

7. The method of claim 1 or 2, wherein the testing comprises:
testing the biopsy sample with a microarray to determine the amount of HER-1 in the biopsy sample.

8. The method of claim 1 or 2, wherein the testing comprises:
testing the biopsy sample with a microarray to determine the amount of ERBB2 in the biopsy sample.

9. The method of claim 1 or 2, wherein the testing comprises:
testing the biopsy sample with a microarray to determine the amount of vascular endothelial growth factor receptor (VEGFR) in the biopsy sample.

10. A method for treating a patient with a breast tumor comprising:
inserting a cryoprobe or cryobiopsy device into the breast of the patient and into the tumor;
freezing a biopsy sample within the tumor;
excising the frozen biopsy sample from the from the breast, after the biopsy sample is frozen;
testing the biopsy sample for the presence of a signaling substance which indicates the susceptibility of the tumor to a specific pharmaceutical treatment;
administering the specific pharmaceutical treatment to the patient.

11. A method for treating a patient with a breast tumor comprising:
inserting a cryoprobe or cryobiopsy device into the breast of the patient and into the tumor;
freezing a biopsy sample within the tumor;
excising the frozen biopsy sample from the from the breast, after the biopsy sample is frozen;
testing the biopsy sample for the presence of a signaling substance which indicates the prognosis of the tumor;
administering a treatment to the patient based on the prognosis.

12. The method of claim 10 or 11, wherein:
the testing comprises testing the biopsy sample with a microarray to determine the amount of mRNA in the biopsy sample, and determining if the amount or type of mRNA is abnormal; and
the treatment comprises, if the amount or type of mRNA is abnormal, administering chemotherapy to the patient.

13. The method of claim 10 or 11, wherein:
the testing comprises testing the biopsy sample with a microarray to determine the amount of mRNA in the biopsy sample, and determining if the amount or type of mRNA is abnormal; and
the treatment comprises, if the amount or type of mRNA is abnormal, administering a targeted therapy.

14. The method of claim 10 or 11, wherein:
the testing comprises testing the biopsy sample with a microarray to determine the amount of mutant phosphorylating mRNA in the biopsy sample, and determining if the amount or type of mRNA is abnormal; and
the treatment comprises, if the amount or type of mRNA is abnormal, administering Iressa to the patient.

15. The method of claim 10 or 11, wherein:
the testing comprises testing the biopsy sample with a microarray to determine the amount of mRNA in the biopsy sample, and determining if the amount of mRNA exceeds a predetermined amount; and
the treatment comprises, if the level of mRNA is above the predetermined limit, administering Iressa.

16. The method of claim 10 or 11, wherein the testing comprises:
testing the biopsy sample with immunohistochemistry to determine the amount of HER-2/Neu in the biopsy sample, and determining if the amount of HER-2/Neu exceeds a predetermined amount; and
the treatment comprises, if the level of HER-2/Neu is above the predetermined limit, administering Herceptin.

17. The method of claim 10 or 11, wherein the testing comprises:
testing the biopsy sample with immunohistochemistry to determine the amount of estrogen or progesterone receptors in the biopsy sample, and determining if the amount of estrogen or progesterone receptors exceeds a predetermined amount; and
the treatment comprises, if the level of estrogen or progesterone receptors is above the predetermined limit, administering hormonal therapy.

18. The method of claim 10 or 11, wherein the testing comprises:
testing the biopsy sample with a microarray to determine the amount of VEGFR in the biopsy sample.

19. The method of claim 10 or 11, wherein the testing comprises:
testing the biopsy sample with a microarray to determine the amount of ERBB2 in the biopsy sample.

20. A system for diagnosing cancer in a patient, said system comprising:
means for freezing a biopsy sample in situ within the patient's body, said means for freezing being operable to cool the biopsy sample to cryopreserving temperatures;
means for excising the biopsy sample from the patient's body while the biopsy sample is frozen;
means for maintaining the biopsy sample in a frozen state;
means for testing the biopsy sample to determine the over-expression of a signaling substance within the biopsy sample.

21. A system for diagnosing cancer in a patient, said system comprising:
a cryoprobe for freezing a biopsy sample in situ within the patient's body, said cryoprobe being operable to cool the biopsy sample to cryopreserving temperatures;
means for excising the biopsy sample from the patient's body while the biopsy sample is frozen;
means for maintaining the biopsy sample in a frozen state;
a microarray test kit for testing the biopsy sample to determine the abnormal expression of a signaling substance within the biopsy sample.

22. A system for diagnosing cancer in a patient, said system comprising:
a cryobiopsy device for freezing a biopsy sample in situ within the patient's body, said cryobiopsy device being operable to cool the biopsy sample to cryopreserving temperatures;
means for excising the biopsy sample from the patient's body while the biopsy sample is frozen;
means for maintaining the biopsy sample in a frozen state;
a microarray test kit for testing the biopsy sample to determine the abnormal expression of a signaling substance within the biopsy sample.
